(19) United States
(12) Patent Application Publication
(10) Pub. No.: US 2024/0211907 A1
(43) Pub. Date: Jun. 27, 2024

(54) METHODS AND SYSTEMS FOR DYNAMICALLY DETERMINIG TRANSACTION VARIABLES BASED ON CONTINUOUS MONITORING OF DATA SOURCES

(71) Applicant: Optum, Inc., Minnetonka, MN (US)

(72) Inventor: Joshua R. BARTELME, Minneapolis, MN (US)

(73) Assignee: Optum, Inc., Minnetonka, MN (US)

(21) Appl. No.: 18/146,660

(22) Filed: Dec. 27, 2022

(51) Int. Cl. G06Q 20/10; G06Q 20/38; G16H 20/10

(52) U.S. Cl. CPC G06Q 20/102; G06Q 20/387; G16H 20/10

(57) ABSTRACT

A method can include receiving prescription information identifying a prescription for an account to be filled in a recurring manner at multiple fill time points; receiving payment option information; receiving a single selection indicating a standing instruction to fill the prescription for each of the multiple fill time points at a respective lowest payment amount for the account regardless of a plurality of payment options; based on the single selection and for each of the multiple fill time points: obtaining, from a plurality of external data sources via a network, a plurality of payment amounts, determining the respective lowest payment amount for the respective fill time point among the plurality of payment amounts, selecting a payment option among the plurality of payment options associated with the respective lowest payment amount for the respective fill time point, and causing the prescription to be filled using the selected payment option.

### TECHNICAL FIELD

The present disclosure relates to computer-implemented methods and systems for dynamically determining transaction variables based on monitoring data sources continuously.

### BACKGROUND

A patient can obtain a prescription from a medical professional, and can contract with a service provider to fill the prescription. The prescription can be filled in a recurring manner at various time points (e.g., weekly, bi-weekly, monthly, etc.).

In some cases, the service provider selects a particular payment option, and processes the filling of the prescription according to the selected payment option. As an example, a service provider can select to use the patient's insurance as a payment option, and can charge the patient a payment amount that is associated with the patient's insurance. In such cases, the service provider might not notify the patient of alternative, or supplemental, payment options. Moreover, the selected payment option might not provide the lowest payment amount to the patient.

In other cases, a patient can select a particular payment option, and a service provider can process the prescription according to the selected payment option. For instance, a patient can attempt to research the respective payment amounts of various payment options in order to compare the respective payment amounts. However, the payment amount information might be unavailable or inaccessible to the patient. In some instances, the patient might ascertain a payment amount of a payment option, and might desire to utilize that payment option to fill the prescription. In such cases, the patient can then be required to find a particular service provider that can fill the prescription using that payment option.

In the event that a patient selects a particular payment option to be used to fill a prescription, the selected payment option can be used by the service provider in all subsequent fills. If the patient desires to change the payment option, then the user might be required to instruct the service provider to change the payment option, which might be inefficient and impractical.

Moreover, the payment amounts of various prescriptions frequently fluctuates across fill time points. Accordingly, a particular payment option might yield the lowest payment amount for a patient at one fill time point, and another payment option might yield the lowest payment amount for the patient at another temporally close fill time point.

The foregoing concerns and impracticalities become more acute in the event that the patient has multiple prescriptions. Further, the foregoing concerns and impracticalities become even more acute in the event that the multiple prescriptions have different, or staggered, fill time points.

Accordingly, a need exists for an improved user experience and user interface for prescription filling that is efficient, inexpensive, less error-prone, and less computationally intensive.

### SUMMARY

According to an embodiment of the present disclosure, a computer-implemented method can include receiving, by one or more processors, prescription information identifying a prescription for an account to be filled in a recurring manner at multiple fill time points; receiving, by the one or more processors, payment option information identifying a plurality of payment options of the account for the prescription; receiving, by the one or more processors, a single selection indicating a standing instruction to fill the prescription for each of the multiple fill time points at a respective lowest payment amount for the account regardless of the plurality of payment options; and based on the single selection and for each of the multiple fill time points: obtaining, by the one or more processors and from a plurality of external data sources via a network, a plurality of payment amounts of respective ones of the plurality of payment options of the account for the respective fill time point, wherein the plurality of payment amounts fluctuate across the multiple fill time points, determining, by the one or more processors, the respective lowest payment amount for the respective fill time point among the plurality of payment amounts, selecting, by the one or more processors, a payment option among the plurality of payment options associated with the respective lowest payment amount for the respective fill time point, and causing, by the one or more processors, the prescription to be filled using the selected payment option.

According to another embodiment of the present disclosure, a device can include a memory configured to store instructions; and a processor configured to execute the instructions to perform operations comprising: receiving prescription information identifying a prescription for an account to be filled in a recurring manner at multiple fill time points; receiving payment option information identifying a plurality of payment options of the account for the prescription; receiving a single selection indicating a standing instruction to fill the prescription for each of the multiple fill time points at a respective lowest payment amount for the account regardless of the plurality of payment options; and based on the single selection and for each of the multiple fill time points: obtaining, from a plurality of external data sources via a network, a plurality of payment amounts of respective ones of the plurality of payment options of the account for the respective fill time point, wherein the plurality of payment amounts fluctuate across the multiple fill time points, determining the respective lowest payment amount for the respective fill time point among the plurality of payment amounts, selecting a payment option among the plurality of payment options associated with the respective lowest payment amount for the respective fill time point, and causing the prescription to be filled using the selected payment.

According to another embodiment of the present disclosure, a non-transitory computer-readable medium can store instructions that, when executed by a processor, cause the processor to perform operations comprising: receiving prescription information identifying a prescription for an account to be filled in a recurring manner at multiple fill time points; receiving payment option information identifying a plurality of payment options of the account for the prescription; receiving a single selection indicating a standing instruction to fill the prescription for each of the multiple fill time points at a respective lowest payment amount for the account regardless of the plurality of payment options; and based on the single selection and for each of the multiple fill time points: obtaining, from a plurality of external data sources via a network, a plurality of payment amounts of respective ones of the plurality of payment options of the account for the respective fill time point, wherein the plurality of payment amounts fluctuate across the multiple fill time points, determining the respective lowest payment amount for the respective fill time point among the plurality of payment amounts, selecting a payment option among the plurality of payment options associated with the respective lowest payment amount for the respective fill time point, and causing the prescription to be filled using the selected payment.

The embodiments of the present disclosure permit a platform to cause a prescription to be filled for multiple fills using a respective payment option that yields a lowest payment amount for an account based on a single selection indicating a standing instruction to fill the prescription for each of the fills. In this way, the embodiments herein provide an improved user interface that permits a patient (or another user) to provide a single selection that is applied to multiple fills instead of requiring the patient to select a payment option at each fill time point. The payment amounts of the respective payment options fluctuate across fills. In other words, a first payment option can be associated with the lowest payment amount for a first fill, and a second payment option can be associated with the lowest payment amount for a second, subsequent, fill. Because the payment amounts are dynamic and can fluctuate over time, a particular payment option might not always be associated with the lowest payment amount. However, the platform disclosed in the present disclosure can nonetheless fill the prescription using a lowest payment amount to the account based on the single selection indicating the standing instruction, by continuously monitoring external data sources for payment amounts that fluctuate across fill time points and automatically calculating payments amounts associated with different payment options for a particular fill point based on the payment amounts obtained from the external data sources for that particular fill point. In this way, some implementations herein reduce an amount of required selections, which thereby conserves processor and memory resources of devices associated with prescription filling.

The embodiments of the present disclosure permit the platform to obtain payment amounts of the payment options at each fill time point. As mentioned above, the particular payment option associated with the lowest payment amount can vary on a daily, weekly, monthly, etc., basis because of the dynamic fluctuations of the payment amounts of the payment options. The platform can automatically determine the payment amounts at each fill time point instead of requiring the patient to attempt to ascertain such information. In this way, some implementations herein reduce an amount of required processing, which thereby conserves processor and memory resources of devices associated with prescription filling.

It can be understood that both the foregoing general description and the following detailed description are examples and explanatory only and are not restrictive of the embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIGS. 1A-1D are diagrams of an example implementation for automatically causing a prescription to be filled at a lowest payment amount for an account regardless of payment options.

FIG. 2 is a diagram of an example environment for automatically causing a prescription to be filled at a lowest payment amount for an account regardless of payment options.

FIG. 3 is a diagram of components of one or more devices of FIG. 2.

FIG. 4 is a flowchart of an example process for automatically causing a prescription to be filled at a lowest payment amount for an account regardless of payment options.

FIGS. 5A-5G are diagrams of example user interfaces.

### DETAILED DESCRIPTION

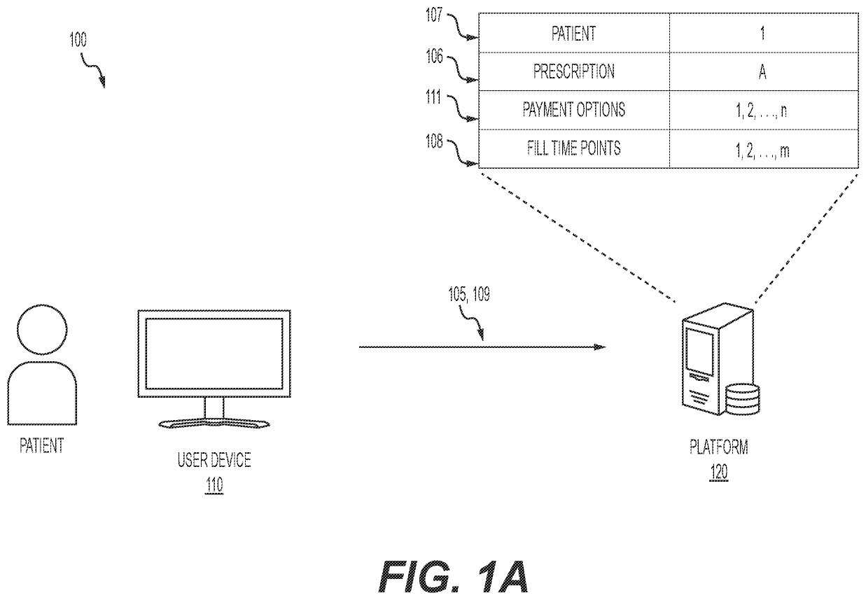

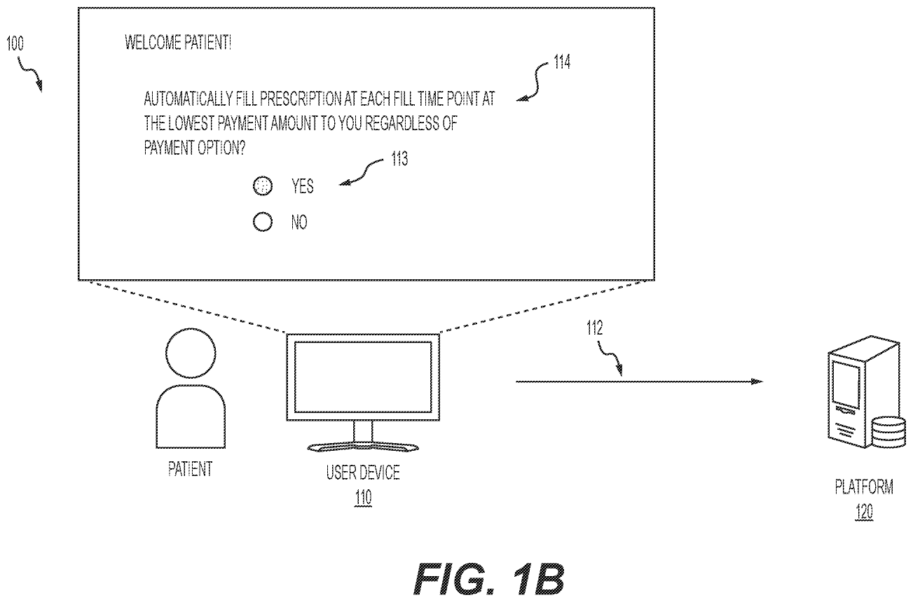

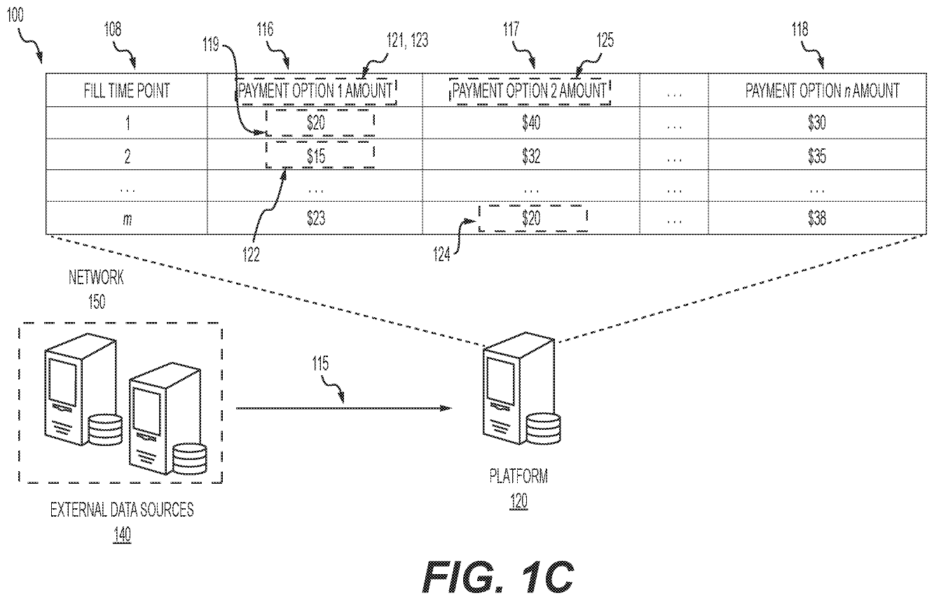

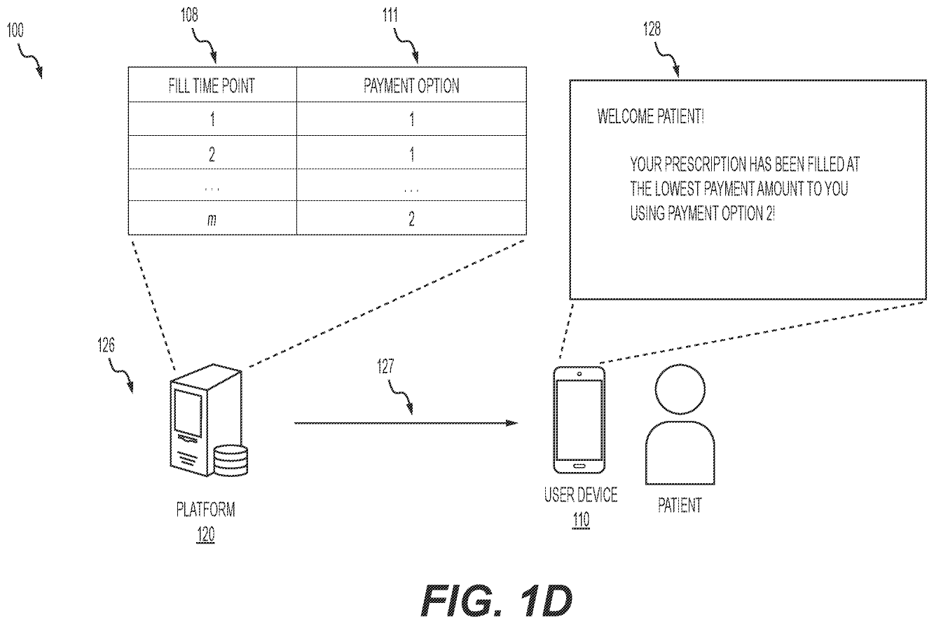

FIGS. 1A-1D are diagrams of an example implementation 100 for automatically causing a prescription to be filled at a lowest payment amount for an account regardless of payment options.

As shown in FIG. 1A, a platform 120 (e.g., a server) can receive 105, from a user device 110 (e.g., a desktop computer, a laptop, a smartphone, or the like), prescription information identifying a prescription 106 for an account 107 that is to be filled in a recurring manner at fill time points 108. The prescription information can identify the account 107 (e.g., “Account 1”), and identify a prescription 106 of the account (e.g., “Prescription A”). Further, the prescription information can identify fill time points 108 of the prescription 106 (e.g., “fill time point 1,” “fill time point 2,” . . . “fill time point m”). The platform 120 can also receive 109 payment option information identifying payment options 111 for the prescription 106. The information identifying the payment options can identify payment options 111 that can be used to pay for the prescription 106 (e.g., “payment option 1,” “payment option 2,” . . . “payment option n”). For example, the payment options 111 can be insurance (“on-benefit”), non-insurance (“off-benefit”), cash, coupons, discount cards, prescription savings programs, drug saving charities, or the like. While FIG. 1A identifies the user as a patient, the user can additionally or alternatively be a health care provider communicating with the platform 120 using the user device 110. For example, the platform 120 can receive all or a portion of the patient identification information, prescription information, and payment option identification information from a health care provider.

As shown in FIG. 1B, the platform 120 can receive 112, from the user device 110, a single selection 113 indicating a standing instruction to fill the prescription 106 for each of the fill time points 108 at a lowest payment amount for the account regardless of the payment options 111. The user device 110 can provide a prompt 114 for the patient that permits the patient to select the prescription 106 to be automatically filled at each fill time point 108 at the lowest payment amount for the account 107 (e.g., “Automatically fill prescription at each fill time point at the lowest payment amount to you regardless of payment option”), and can receive a single selection 113 (e.g., “Yes”) based on the prompt 114. The user device 110 can provide the single selection 113 to the platform 120.

As shown in FIG. 1C, the platform 120 can obtain or receive 115, from external data sources 140 via a network 150, payment amounts 116, 117, and 118 of the various payment options 111 of the account 107 for the prescription 106. The platform 120 can, for each of the fill time points 108, determine respective payment amounts 116, 117, and 118 of the payment options 111 based on the obtained payment amounts. As an example, for fill time point 1, the platform 120 can determine a payment amount of $20 for payment option 1, determine a payment amount of $40 for payment option 2, and determine a payment amount of $30 for payment option n. Further, for fill time point 2, the platform 120 can determine a payment amount of $15 for payment option 1, determine a payment amount of $32 for payment option 2, and determine a payment amount of $35 for payment option n. Further still, for fill time point m, the platform 120 can determine a payment amount of $23 for payment option 1, determine a payment amount of $20 for payment option 2, and determine a payment amount of $38 for payment option n.

The platform 120 can, for each of the fill time points 108, determine the lowest payment amount for the account 107 based on the respective payment amounts 116, 117, and 118 of the payment options 111, and select a payment option associated with the lowest payment amount for the account 107. For example, the platform 120 can determine 119 that $20 is the lowest payment amount for the patient for fill time point 1, and select 121 payment option 1 that is associated with that payment amount. As another example, the platform 120 can determine 122 that $15 is the lowest payment amount for the patient for fill time point 2, and select 123 payment option 1 that is associated with that payment amount. As yet another example, the platform 120 can determine 124 that $20 is the lowest payment amount for fill time point m, and select 125 payment option 2 that is associated with that payment amount.

As shown in FIG. 1D, the platform 120 can, for each of the fill time points 108, automatically cause 126 the prescription to be filled using the payment option 111 associated with the lowest payment amount for the account 107, based on the single selection 113 indicating the standing instruction to fill the prescription 106 for each of the fill time points 108 at the lowest payment amount for the account 107 regardless of the payment options 111. For example, for fill time point 1, the platform 120 can automatically cause 126 the prescription to be filled using payment option 1. For fill time point 2, the platform 120 can automatically cause 126 the prescription to be filled using payment option 1. For fill time point m, the platform 120 can automatically cause 126 the prescription to be filled using payment option 2. The platform 120 can cause 126 the prescription to be filled at each fill time point 108 based on the single selection 113.

The platform 120 can provide 127 a notification to the user device 110 (e.g., of the patient) that identifies that the prescription 106 has been automatically filled using a payment option associated with the lowest payment amount for the account. For instance, the user device 110 can display 128, for fill time point m, the notification indicating that “Your prescription has been filled at the lowest payment amount to you using payment option 2!” Additionally, the platform 120 can provide a notification to a user device 110 (e.g., of the patient) at a predetermined time before the prescription is automatically filled, indicating that the prescription will be automatically filled using a payment option associated with the lowest payment amount for the account. Also, the same or a separate notification can include information on payment amounts associated with different payment options that can be applicable to the upcoming refill. The patient can be provided an option to select from the different payment options, at which point the automatic filling of the upcoming refill can be disabled and the selected payment option can be applied to the upcoming refill. In this way, the platform 120 can be able to provide more flexibility in selecting a payment option, in case a non-lowest cost payment option can provide a certain benefit to the patient different from the reduced payment amount (e.g., accumulation of redeemable points, etc.).

In this way, the embodiments herein improve patient convenience and experience associated with prescription filling by permitting a user to provide a single selection via a user interface indicating a standing instruction to fill the prescription using a payment option that yields a lowest payment amount for the account. In this way, some embodiments herein conserve computational resources of devices associated with prescription filling by reducing a number of user inputs and/or messaging required for prescription filling. Moreover, in this way, some embodiments herein prevent, or reduce, the need of a patient to research payment amounts for different payment options, instruct various service providers to fill prescriptions using the various payment options, and/or travel to various service provider locations to fill prescriptions. As such, some embodiments herein improve patient experience, patient convenience, and patient safety.

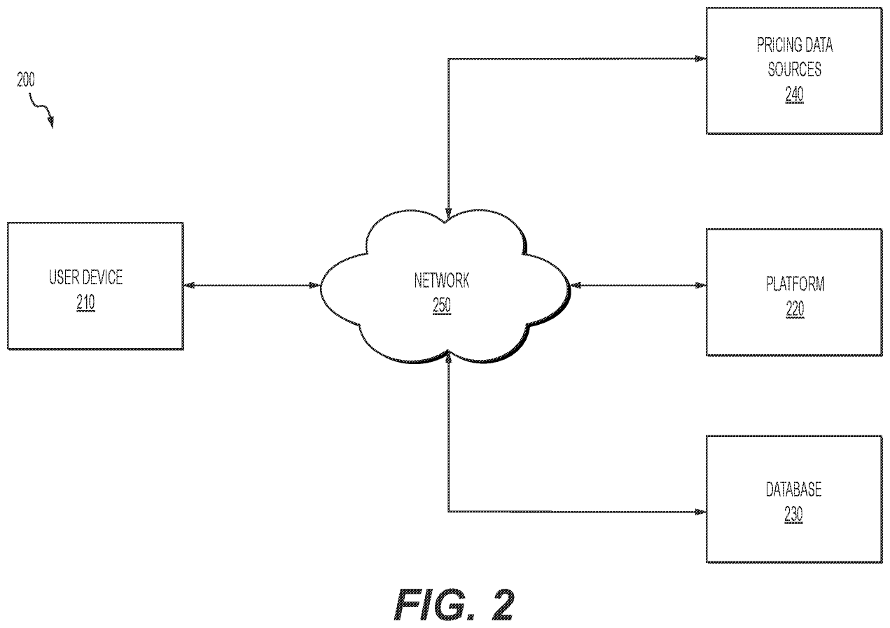

FIG. 2 is a diagram of an example environment 200 for automatically causing a prescription to be filled at a lowest payment amount for a patient regardless of payment options. As shown in FIG. 2, the environment 200 can include a user device 210, a platform 220, a database 230, external data sources 240, and a network 250. The user device 210, the platform 220, the database 230, the external data sources 240, and the network 250 can correspond, respectively, to the user device 110, the platform 120, the database 130, the external data sources 140, and the network 150 shown in FIGS. 1A-1D.

User device 210 can be a device configured to provide information identifying a prescription of an account that is to be filled in a recurring manner at fill time points; provide payment option information identifying payment options for the prescription; and provide a single selection indicating a standing instruction to fill the prescription for each of the fill time points at a lowest payment amount for the account regardless of the payment options. For example, the user device 210 can be a smartphone, a desktop computer, a laptop computer, a tablet computer, a wearable device, or the like.

Platform 220 can be a device configured to receive prescription information identifying a prescription of a patient that is to be filled in a recurring manner at fill time points; receive payment option information identifying payment options for the prescription; receive a single selection indicating a standing instruction to fill the prescription for each of the fill time points at a lowest payment amount for the account regardless of the payment options; based on the single selection indicating the standing instruction and for each of the fill time points, determine respective payment amounts of the payment options; based on the single selection indicating the standing instruction and for each of the fill time points, determine the lowest payment amount for the account based on the respective payment amounts of the payment options; based on the single selection indicating the standing instruction and for each of the fill time points, select a payment option associated with the lowest payment amount for the account; and based on the single selection indicating the standing instruction and for each of the fill time points, automatically cause the prescription to be filled using the selected payment option associated with the lowest payment amount for the patient. For example, the platform 220 can be a server, a computer, or the like.

Database 230 can be a device configured to store prescription information identifying a prescription of an account that is to be filled in a recurring manner at fill time points; store payment options for the prescription; store a single selection indicating a standing instruction to fill the prescription for each of the fill time points at a lowest payment amount for the patient regardless of the payment options; store respective payment amounts of the payment options; store the lowest payment amount for the patient based on the respective payment amounts of the payment options; store a payment option associated with the lowest payment amount for the patient; and store information identifying a savings for the account for each fill time point. For example, the database 230 can be a database, a computer, or the like.

External data sources 240 can be devices configured to store respective payment amounts of respective payment options. For example, external data sources 240 can be databases, servers, computers, or the like. As discussed above, the payment amounts of the respective payment options may fluctuate over time (e.g., across fill time points 108). Platform 220 may continuously monitor the external data sources 240 to obtain accurate payment amounts for the different payment options at any given time.

Network 250 can be a cellular network (e.g., a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, or the like, and/or a combination of these or other types of networks.

The number and arrangement of the devices of the environment 200 shown in FIG. 2 are provided as an example. In practice, the environment 200 can include additional devices, fewer devices, different devices, or differently arranged devices than those shown in FIG. 2. Additionally, or alternatively, a set of devices (e.g., one or more devices) of the environment 200 can perform one or more functions described as being performed by another set of devices of the environment 200.

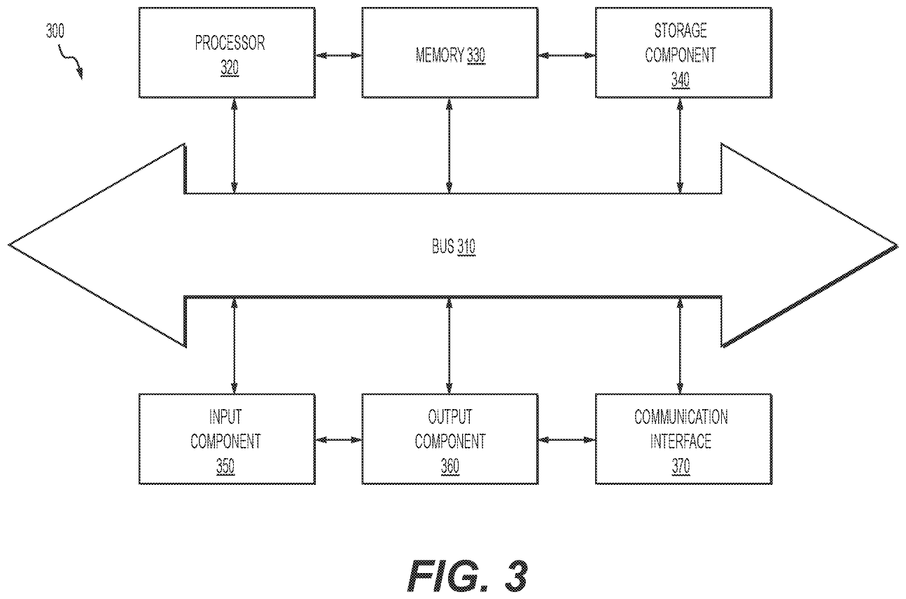

FIG. 3 is a diagram of components 300 of one or more devices of FIGS. 1A-1D and/or FIG. 2. The components 300 can include a bus 310, a processor 320, a memory 330, a storage component 340, an input component 350, an output component 360, and a communication interface 370.

The bus 310 includes a component that permits communication among the components 300. The processor 320 can be implemented in hardware, firmware, or a combination of hardware and software. The processor 320 can be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 320 can include one or more processors capable of being programmed to perform a function. The memory 330 can include a random access memory (RAM), a read only memory (ROM), and/or another type of dynamic or static storage device (e.g., a flash memory, a magnetic memory, and/or an optical memory) that stores information and/or instructions for use by the processor 320.

The storage component 340 can store information and/or software related to the operation and use of the components 300. For example, the storage component 340 can include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 350 can include a component that permits the components 300 to receive information, such as via user input (e.g., a touch screen display, a keyboard, a keypad, a mouse, a button, a switch, and/or a microphone for receiving the reference sound input). Additionally, or alternatively, the input component 350 can include a sensor for sensing information (e.g., a global positioning system (GPS) component, an accelerometer, a gyroscope, and/or an actuator). The output component 360 can include a component that provides output information from the components 300 (e.g., a display, a speaker for outputting sound at the output sound level, and/or one or more light-emitting diodes (LEDs)).

The communication interface 370 can include a transceiver-like component (e.g., a transceiver and/or a separate receiver and transmitter) that enables the components 300 to communicate with other devices, such as via a wired connection, a wireless connection, or a combination of wired and wireless connections. The communication interface 370 can permit the components 300 to receive information from another device and/or provide information to another device. For example, the communication interface 370 can include an Ethernet interface, an optical interface, a coaxial interface, an infrared interface, a radio frequency (RF) interface, a universal serial bus (USB) interface, a Wi-Fi interface, a cellular network interface, or the like.

The components 300 can perform one or more processes described herein. The components 300 can perform these processes based on the processor 320 executing software instructions stored by a non-transitory computer-readable medium, such as the memory 330 and/or the storage component 340. A computer-readable medium can be defined herein as a non-transitory memory device. A memory device can include memory space within a single physical storage device or memory space spread across multiple physical storage devices.

The software instructions can be read into the memory 330 and/or the storage component 340 from another computer-readable medium or from another device via the communication interface 370. When executed, the software instructions stored in the memory 330 and/or the storage component 340 can cause the processor 320 to perform one or more processes described herein. Additionally, or alternatively, hardwired circuitry can be used in place of or in combination with software instructions to perform one or more processes described herein. Thus, implementations described herein are not limited to any specific combination of hardware circuitry and software.

The number and arrangement of the components shown in FIG. 3 are provided as an example. In practice, the components 300 can include additional components, fewer components, different components, or differently arranged components than those shown in FIG. 3. Additionally, or alternatively, a set of components 300 (e.g., one or more components) can perform one or more functions described as being performed by another set of components 300.

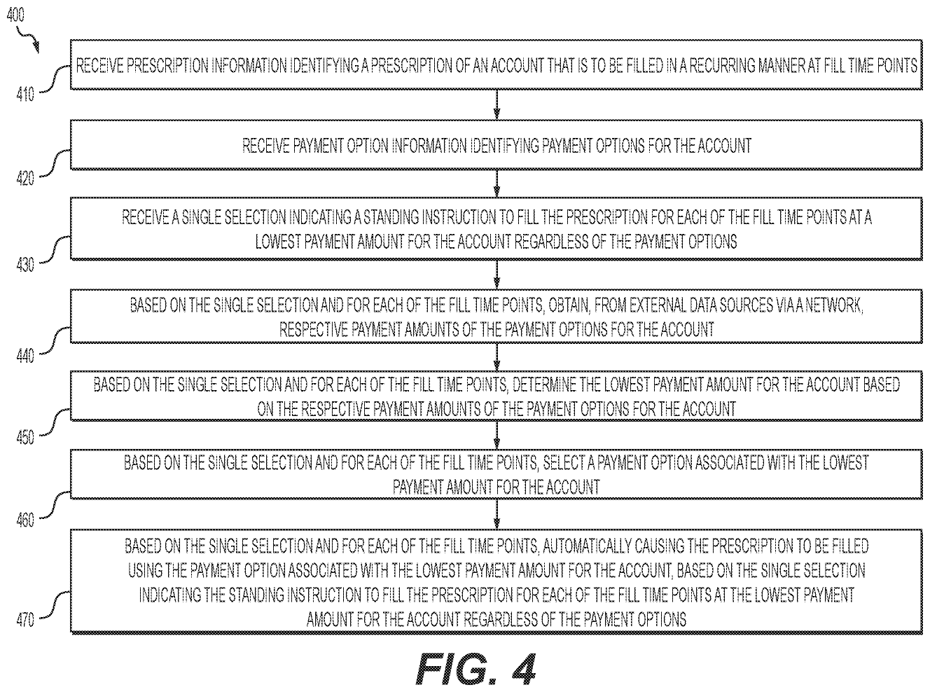

FIG. 4 is a flowchart of an example process 400 for automatically causing a prescription to be filled at a lowest payment amount for an account regardless of payment options.

As shown in FIG. 4 at block 410, the process 400 can include receiving prescription information identifying a prescription of an account that is to be filled in a recurring manner at fill time points (e.g., at event 105).

The prescription information can include an identifier of the account (e.g., an account identifier, a name, a patient identifier, a username, a social security number, etc.). Further, the prescription information can include an identifier of a prescription (e.g., a prescription identifier, name, or the like). Further still, the prescription information can include an identifier of a medical item or service associated with the prescription. For example, the medical item can be a drug, a medication, a medicine, a health service, medical equipment, or the like. The identifier of the medical item or service can be a drug identifier (e.g., a national drug code (NDC), or the like). The prescription information can identify a fill frequency of fill time points (e.g., daily, weekly, bi-weekly, monthly, etc.). Additionally, or alternatively, the prescription information can identify discrete fill time points (e.g., specific calendar dates). The prescription can be filled in a recurring manner at the fill time points.

In some implementations, the platform 220 can receive the prescription information from an external device, such as the user device 210. For example, the patient can interact with the user device 210 to upload the prescription information to the platform 220. In other implementations, the platform 220 can receive some, or all, of the prescription information from another external device, such as the database 230 or an external server. The platform 220 can store the prescription information in the database 230, and obtain the prescription information from the database 230 in subsequent fills of the prescription.

As shown in FIG. 4, at block 420, the process 400 can include receiving payment option information identifying payment options of the account for the prescription (e.g., at event 109).

The payment option information can identify the potential payment options that might be used to pay for the prescription. For example, the payment options can include insurance (“on-benefit”), non-insurance (“off-benefit”), coupons, discount cards, prescription savings programs, drug saving charities, or the like.

The platform 220 can receive the prescription information from an external device, such as the user device 210. For example, the patient can interact with the user device 210 to upload the payment option information to the platform 220. Alternatively, the platform 220 can receive some, or all, of the payment option information from another external device, such as the database 230 or an external server. The platform 220 can store the payment option information in the database 230, and obtain the payment option information from the database 230 in subsequent fills of the prescription.

As shown in FIG. 4 at block 430, the process 400 can include receiving a single selection indicating a standing instruction to fill the prescription for each of the fill time points at a lowest payment amount for the account regardless of the payment options (e.g., at block 112).

The platform 220 can provide a prompt to the user device 210, and the user device 210 can display the prompt. For example, the prompt can request the patient whether the patient would like to select the prescription to be filled for each of the fill time points at a lowest payment amount for the patient regardless of the payment options. The patient can interact with the user device 210 to respond to the prompt, such as by selecting the prescription to be filled for each of the fill time points at a lowest payment for the account regardless of the payment options or not selecting the prescription to be filled for each of the fill time points at a lowest payment amount for the patient regardless of the payment options.

The single selection can cause the platform 220 to automatically cause the prescription to be filled at each fill time point at the lowest payment amount to the account regardless of the payment options. For example, based on the single selection via a user interface of the user device 210, the platform 220 can automatically cause the prescription to be filled at each fill time point at a lowest payment amount to the account using a particular payment option that yields the lowest payment amount. Accordingly, some implementations herein provide an improved user interface that permits the patient to provide a single selection that causes one or more subsequent fills of the prescription to be performed at a lowest cost to the patient.

As shown in FIG. 4 at block 440, the process 400 can include, for each of the fill time points, obtaining, from the external data sources 240 via the network 250, payment amounts of the payment options for the account (e.g., at event 115).

The platform 220 can obtain the payment amounts based on respective requests to the external data sources 240. For example, the platform 220 can provide a request to an external data source 240 using a particular communication technique or protocol, and receive a payment amount from the external data source 240 based on the request. The platform 220 can identify the particular external data sources 240 to which to provide requests based on the payment option information. For example, the payment option information can include information identifying, or that permits the platform 220 to identify, device identifiers of the external data sources 240.

The platform 220 can determine respective payment amounts of the payment options. For example, the platform 220 can determine the available payment options based on the payment option information, and determine respective payment amounts of the available payment options. The platform 220 can obtain the payment amounts of the payment options from the external data sources 240.

The platform 220 can determine the respective payment amounts of the payment options for each fill time point. For example, the platform 220 can determine the respective payment amounts of the payment options at a particular time based on a fill time point. As examples, the platform 220 can determine the respective payment amounts of the payment options n days (e.g., 5 days, 4 days, etc.) in advance of the fill time point, in substantially real-time during processing of the fill, at a predefined time, or the like.

The platform 220 can determine the respective payment amounts of the payment options based on requesting the payment amounts from the external data sources 240. Alternatively, the platform 220 can determine the respective prices of the payment options based on processing information received from the external data sources 240. For example, the platform 220 can receive information identifying a particular amount by which a payment amount can be reduced by using a payment option, and can determine a payment amount of the payment option based on reducing a payment amount by the particular amount.

In other words, the platform 220 can dynamically determine transaction variables (e.g., payment amounts of the payment options) based on monitoring data sources (e.g., external data sources 240) continuously.

As shown in FIG. 4 at block 450, the process 400 can include, for each of the fill time points, determining the lowest payment amount for the patient based on the respective payment amounts of the payment options (e.g., at one or more events 119, 122, and 124).

The platform 220 can obtain the payment amounts of the payment options, compare the payment amounts of the payment options, and determine a lowest payment amount for the account. For example, the platform 220 can determine the payment option associated with the lowest payment amount.

In other words, the platform 220 can dynamically determine transaction variables (e.g., payment amounts associated with different payment options, and a lowest payment amount of the payment amounts associated with the different payment options) based on monitoring data sources (e.g., external data sources 240) continuously.

As shown in FIG. 4 at block 460, the process 400 can include, for each of the fill time points, selecting a payment option associated with the lowest payment amount for the account (e.g., at one or more events 121, 123, and 125).

The platform 220 can select the payment option associated with the lowest payment amount for the account.

As shown in FIG. 4 at block 470, the process 400 can include, for each of the fill time points, automatically causing the prescription to be filled using the payment option associated with the lowest payment amount for the account, based on the single selection indicating the standing instruction to fill the prescription for each of the fill time points at the lowest payment amount for the patient regardless of the payment options (e.g., at event 126).

In some implementations, the platform 220 can cause the prescription to be filled using the selected payment option. Additionally, or alternatively, the platform 220 can fill the prescription using the selected payment option. For example, the platform 220 can cause the prescription to be filled, such that the medical item associated with the prescription is delivered to the patient, is delivered to a service provider at which the patient can obtain the medical item, is ordered, is billed, is scheduled to be filled, or the like.

In this way, the platform 220 can automatically cause the prescription to be filled at each fill time point using a particular payment option that is associated with the lowest payment amount for the patient at that fill time point, based on a single selection described in reference to block 430.

In some implementations, the platform 220 can override a particular selection. For example, the platform 220 can receive a selection from the user device 210 to fill a prescription using a particular payment option, and override the selection based on another payment option yielding a lower payment amount than the particular payment option. For example, the platform 220 can perform a validation on the selection, and override a particular payment option value corresponding to a payment option parameter included in the input based on determining that another payment option yields a lower payment amount for the account.

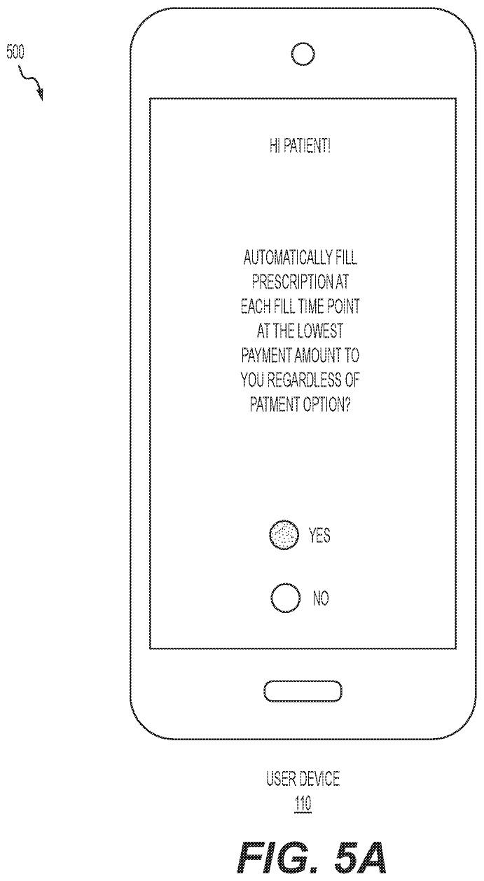

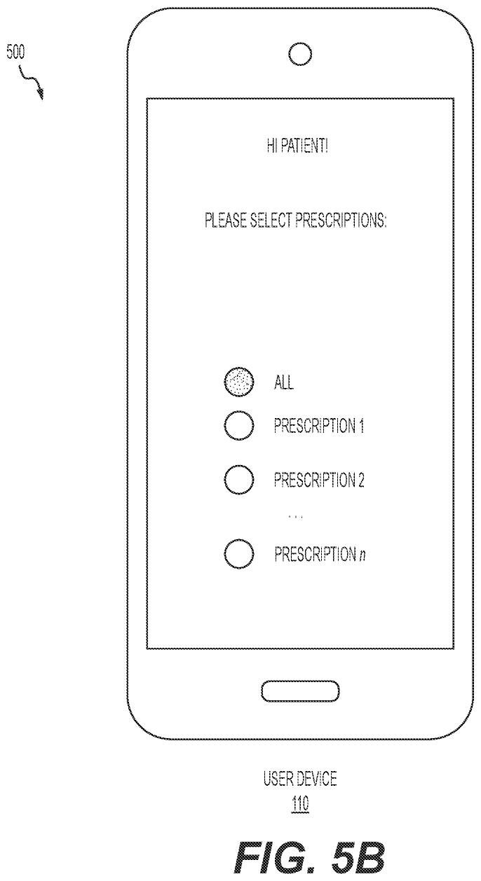

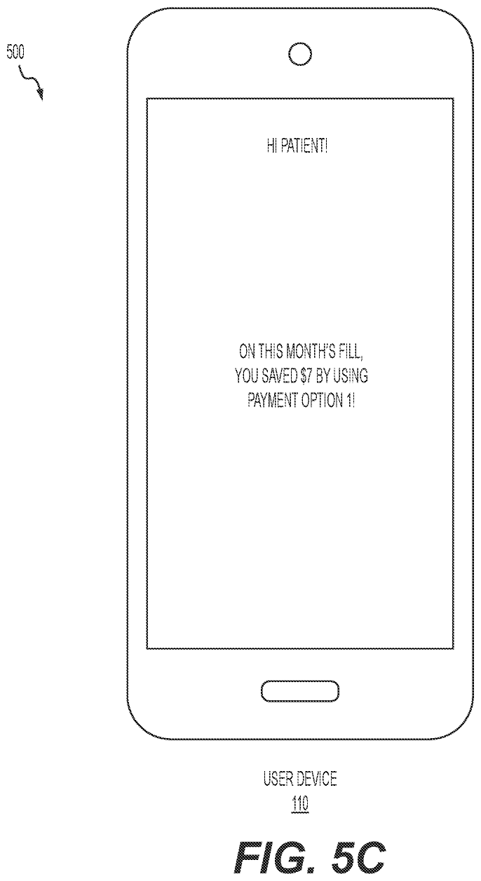

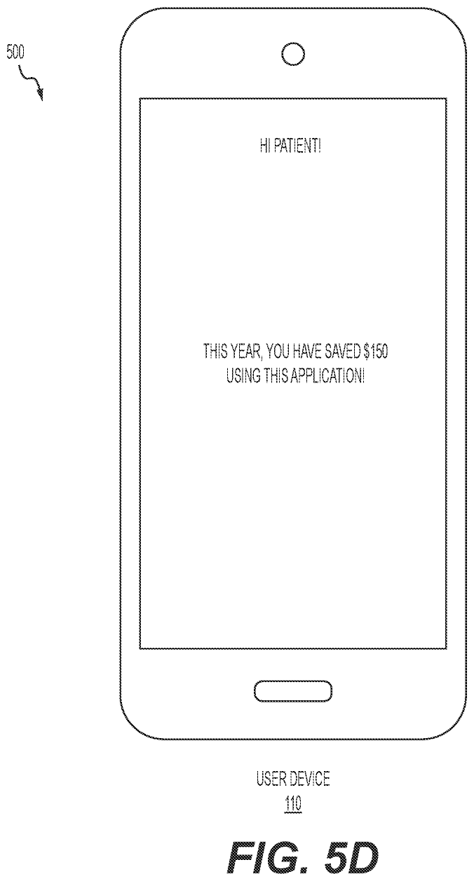

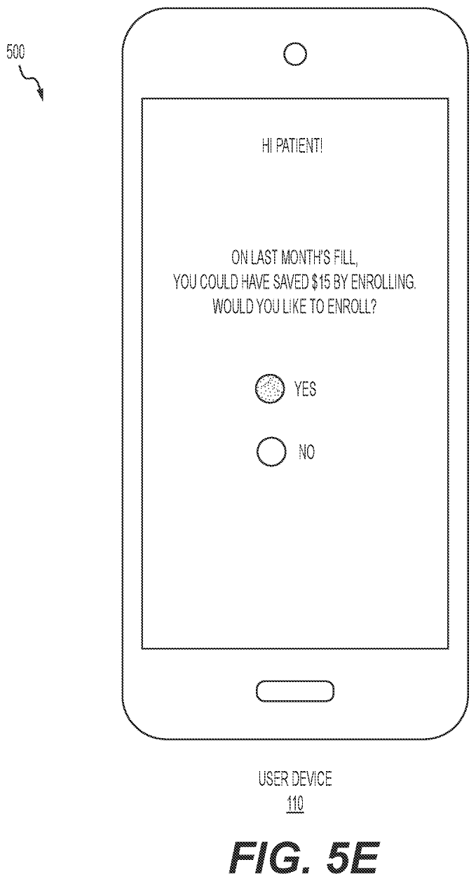

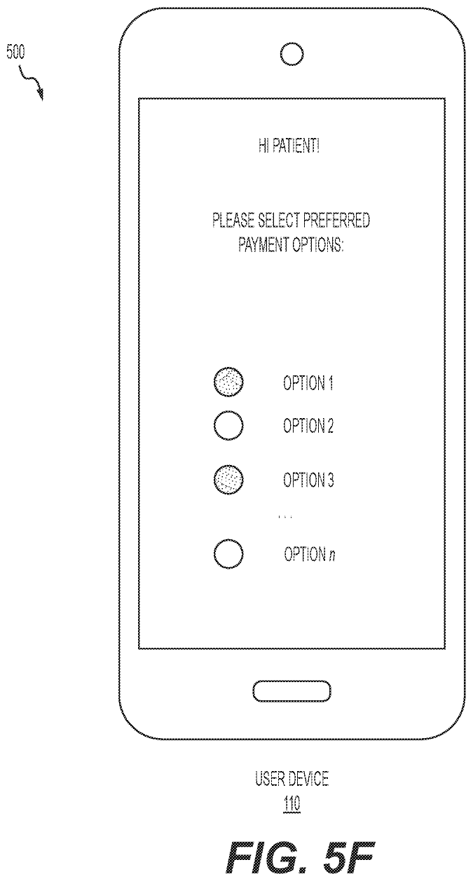

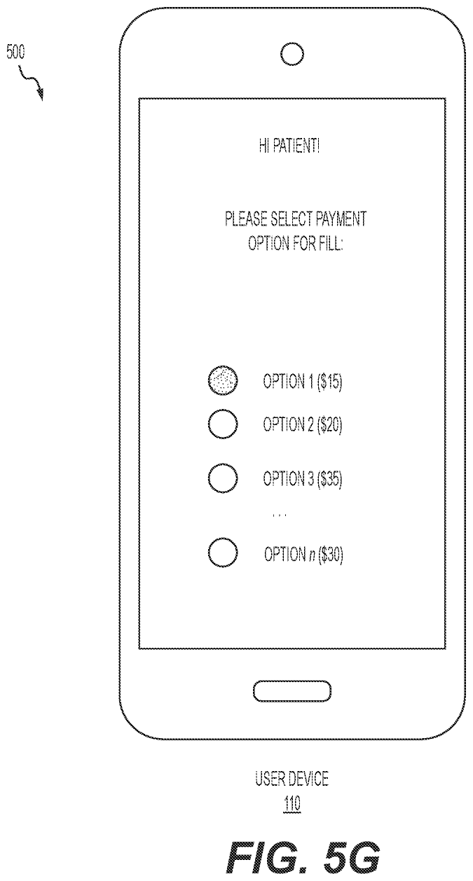

FIGS. 5A-5F are diagrams of example user interfaces 500 of the user device 210.

As shown in FIG. 5A, the user device 210 can display a user interface 500 that prompts the patient to select whether to automatically cause a prescription to be filled at each fill time point at a lowest payment amount to the account regardless of payment option. For example, the user interface 500 can display “Automatically fill prescription at each fill time point at the lowest payment amount to you regardless of payment option?” Further, the user interface 500 can provide selection options for the patient to respond to the prompt. For example, the user interface 500 can display “Yes” or “No.” The user device 210 can provide, to the platform 220, the single selection via the network 250. The platform 220 can automatically fill the prescription at each fill time point at a lowest payment amount to the patient regardless of payment option, based on the single selection indicating the standing instruction that the prescription is to be automatically filled.

As shown in FIG. 5B, the user device 210 can display a user interface 500 that prompts the patient to select the particular prescription(s) of the patient for which the platform 220 is to automatically fill at each fill time point at a lowest payment amount to the patient regardless of payment option. For example, the user interface 500 can provide selection options that correspond to the particular prescription(s) of the patient (e.g., “All,” “Prescription 1,” . . . “Prescription n”). The user device 210 can provide, to the platform 220 via the network 250, the selection. The platform 220 can automatically fill each of the selected prescription(s) at each fill time point at a lowest payment amount to the patient regardless of payment option, based on the single selection.

As shown in FIG. 5C, the user device 210 can display a user interface 500 that displays a savings to the patient provided based on the platform 220 automatically filling a prescription at a particular fill time point at a lowest payment amount to the patient regardless of payment option, and displays the particular payment option that was used to fill the prescription. For example, the user interface 500 can display “On this month's fill, you saved $7 by using payment option 1.” In this case, the platform 220 can compare the payment amounts of the payment options, determine a savings associated with a particular fill based on the payment amounts of the payment options (e.g., based on a difference between the highest payment amount and the lowest payment amount, based on a difference between an average payment amount and the lowest payment amount, based on a difference between a payment amount of a payment option and the lowest payment amount, or the like), and provide information identifying the savings to the user device 210 via the network 250. The user device 210 can be configured to display the savings to the patient.

As shown in FIG. 5D, the user device 210 can display a user interface 500 that displays a total savings to the patient provided based on the platform 220 automatically filling a prescription at multiple fill time points at a lowest payment amount to the account regardless of payment option. For example, the user interface 500 displays “This year, you have saved $150 using this application!” In this case, the platform 220 can determine a total savings associated with a set of fills based on the payment amounts of the payment options for the set of fills, and provide information identifying the total savings to the user device 210 via the network 250. The user device 210 can be configured to display the total savings to the patient.

As shown in FIG. 5E, the user device 210 can display a user interface 500 that displays a potential cost savings to the patient, and that prompts the patient to select whether the platform 220 is to automatically fill a prescription at each fill time point at a lowest payment amount to the patient regardless of payment option. For example, the user interface 500 can display “On last month's fill, you could have saved $15 by enrolling. Would you like to enroll?”

As shown in FIG. 5F, the user device 210 can display a user interface 500 that prompts the patient to select preferred payment options. For example, the user interface 500 can display “Please select preferred payment options,” and can display selection items corresponding to the payment options (e.g., “Option 1,” “Option 2,” etc.). The user device 210 can provide, to the platform 220 via the network 250, the patient selection. The platform 220 can select a payment option, from among the selected payment options, that provides a lowest cost to the patient. That is, instead of selecting from all potential available payment options, the platform 220 can select from only the selected payment options.

As shown in FIG. 5G, the user device 210 can display a user interface 500 that displays the available payment options for a fill and the corresponding payment amounts, and that prompts the patient to select a particular payment option to be used to fill the prescription.

Although the embodiments herein describe automatically filling a prescription, it should be understood that the techniques described herein are applicable to other types of transactions involving other types of goods or services.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the embodiments, as claimed, are not to be considered as limited by the foregoing description.

We claim:

1. A computer-implemented method comprising:
receiving, by one or more processors, prescription information identifying a prescription for an account to be filled in a recurring manner at multiple fill time points;
receiving, by the one or more processors, payment option information identifying a plurality of payment options of the account for the prescription;
receiving, by the one or more processors, a single selection indicating a standing instruction to fill the prescription for each of the multiple fill time points at a respective lowest payment amount for the account regardless of the plurality of payment options; and
based on the single selection and for each of the multiple fill time points:
obtaining, by the one or more processors and from a plurality of external data sources via a network, a plurality of payment amounts of respective ones of the plurality of payment options of the account for the respective fill time point, wherein the plurality of payment amounts fluctuate across the multiple fill time points,
determining, by the one or more processors, the respective lowest payment amount for the respective fill time point among the plurality of payment amounts,
selecting, by the one or more processors, a payment option among the plurality of payment options associated with the respective lowest payment amount for the respective fill time point, and
causing, by the one or more processors, the prescription to be filled using the selected payment option.

2. The computer-implemented method of claim 1, wherein the plurality of payment options include at least one of insurance, a cash payment, a coupon, a discount card, a prescription savings program, or a drug saving charity.

3. The computer-implemented method of claim 1, wherein receiving the single selection comprises receiving the single selection from a user device that is configured to display a prompt of the single selection.

4. The computer-implemented method of claim 1, further comprising:
determining, by the one or more processors, a savings associated with a particular fill at a particular fill time point; and
providing, by the one or more processors, information identifying the savings to a user device that is configured to display the savings.

5. The computer-implemented method of claim 1, further comprising:
determining, by the one or more processors, a total savings associated with the multiple fill time points; and
providing, by the one or more processors, information identifying the total savings to a user device that is configured to display the total savings.

6. The computer-implemented method of claim 1, wherein causing the prescription to be filled comprises:
causing, by the one or more processors, the prescription to be filled automatically using the payment option associated with the lowest payment amount, based on the single selection indicating the standing instruction.

7. The computer-implemented method of claim 1, wherein causing the prescription to be filled comprises:
causing, by the one or more processors, the prescription to be filled using a first payment option at a first fill time point based on the single selection; and
causing, by the one or more processors, the prescription to be filled using a second payment option, that is different than the first payment option, at a second fill time point based on the single selection.

8. A device comprising:
memory configured to store instructions; and
one or more processors configured to execute the instructions to perform operations comprising:
receiving prescription information identifying a prescription for an account to be filled in a recurring manner at multiple fill time points;
receiving payment option information identifying a plurality of payment options of the account for the prescription;
receiving a single selection indicating a standing instruction to fill the prescription for each of the multiple fill time points at a respective lowest payment amount for the account regardless of the plurality of payment options; and
based on the single selection and for each of the multiple fill time points:
obtaining, from a plurality of external data sources via a network, a plurality of payment amounts of respective ones of the plurality of payment options of the account for the respective fill time point, wherein the plurality of payment amounts fluctuate across the multiple fill time points,
determining the respective lowest payment amount for the respective fill time point among the plurality of payment amounts,
selecting a payment option among the plurality of payment options associated with the respective lowest payment amount for the respective fill time point, and
causing the prescription to be filled using the selected payment option.

9. The device of claim 8, wherein the plurality of payment options include at least one of insurance, a cash payment, a coupon, a discount card, a prescription savings program, or a drug saving charity.

10. The device of claim 8, wherein receiving the single selection comprises receiving the single selection from a user device that is configured to display a prompt of the single selection.

11. The device of claim 8, wherein the operations further comprise:
determining a savings associated with a particular fill at a particular fill time point; and
providing information identifying the savings to a user device that is configured to display the savings.

12. The device of claim 8, wherein the operations further comprise:
determining a total savings associated with the multiple fill time points; and
providing information identifying the total savings to a user device that is configured to display the total savings.

13. The device of claim 8, wherein causing the prescription to be filled comprises:
causing the prescription to be filled automatically using the payment option associated with the lowest payment amount, based on the single selection indicating the standing instruction.

14. The device of claim 8, wherein causing the prescription to be filled comprises:
causing the prescription to be filled using a first payment option at a first fill time point based on the single selection; and
causing the prescription to be filled using a second payment option, that is different than the first payment option, at a second fill time point based on the single selection.

15. A non-transitory computer-readable medium storing instructions that, when executed by one or more processors, cause the one or more processors to perform operations comprising:
receiving prescription information identifying a prescription for an account to be filled in a recurring manner at multiple fill time points;
receiving payment option information identifying a plurality of payment options of the account for the prescription;
receiving a single selection indicating a standing instruction to fill the prescription for each of the multiple fill time points at a respective lowest payment amount for the account regardless of the plurality of payment options; and
based on the single selection and for each of the multiple fill time points:
obtaining, from a plurality of external data sources via a network, a plurality of payment amounts of respective ones of the plurality of payment options of the account for the respective fill time point, wherein the plurality of payment amounts fluctuate across the multiple fill time points,
determining the respective lowest payment amount for the respective fill time point among the plurality of payment amounts,
selecting a payment option among the plurality of payment options associated with the respective lowest payment amount for the respective fill time point, and
causing the prescription to be filled using the selected payment option.

16. The non-transitory computer-readable medium of claim 15, wherein the plurality of payment options include at least one of insurance, a cash payment, a coupon, a discount card, a prescription savings program, or a drug saving charity.

17. The non-transitory computer-readable medium of claim 15, wherein receiving the single selection comprises receiving the single selection from a user device that is configured to display a prompt of the single selection.

18. The non-transitory computer-readable medium of claim 15, wherein the operations further comprise:
determining a savings associated with a particular fill at a particular fill time point; and
providing information identifying the savings to a user device that is configured to display the savings.

19. The non-transitory computer-readable medium of claim 15, wherein the operations further comprise:
determining a total savings associated with the multiple fill time points; and
providing information identifying the total savings to a user device that is configured to display the total savings.

20. The non-transitory computer-readable medium of claim 15, wherein causing the prescription to be filled comprises:
causing the prescription to be filled automatically using the payment option associated with the lowest payment amount, based on the single selection indicating the standing instruction.
